(19) United States
(12) Patent Application Publication
(10) Pub. No.: US 2011/0104700 A1
(43) Pub. Date: May 5, 2011

(54) MOLECULAR SIGNATURE FOR FIBROSIS AND ATROPHY

(76) Inventor: Philip F. Halloran, Edmonton (CA)

(21) Appl. No.: 12/995,221

(22) PCT Filed: May 28, 2009
(86) PCT No.: PCT/CA09/00697

§ 371 Date: Jan. 20, 2011

Related U.S. Application Data
(60) Provisional application No. 61/057,656, filed on May 30, 2008.

(51) Int. Cl. C12Q 1/68; G01N 33/53

(52) U.S. Cl. 435/6; 435/7.21

(57) ABSTRACT

Materials and Methods involved in assessing tissue fibrosis and atrophy in mammals. For example, materials and methods involved in detecting organ (e.g., kidney) fibrosis/atrophy due to organ rejection are provided, as are materials and methods for determining the extent of fibrosis/atrophy in mammals such as humans, for example.

### CROSS-REFERENCE TO RELATED APPLICATIONS

This application claims benefit of priority from U.S. Provisional Application Ser. No. 61/057,656, filed on May 30, 2008.

### TECHNICAL FIELD

This document relates to methods and materials involved in assessing tissue fibrosis and atrophy (e.g., fibrosis and atrophy induced by organ rejection) in mammals.

### BACKGROUND

Most renal allografts eventually fail. Histologically, allograft deterioration can be assessed by the extent of inflammation, as well as interstitial fibrosis and tubular atrophy (IFTA), which is driven by inflammation and occurs over time. Although histopathology has been a cornerstone of disease assessment and classification for over a century, it has severe limitations. Laboratory testing of tissue samples relies on subjective assessment of stained sections under the microscope. Expert pathologists are in short supply, and clinicians must rely on arbitrary ordinal classifications of lesions that are summarized into dichotomous diagnoses that bear little relationship to function or biology, are subjective and poorly reproducible, and have not been validated against objective external tests because none exist. Therapeutic decisions are based on the histological assessment, and misdiagnosis can lead to under- or over-treatment.

### SUMMARY

This document provides methods and materials involved in assessing tissue fibrosis and atrophy (e.g., fibrosis and atrophy induced by organ rejection) in mammals. For example, this document provides methods and materials involved in early detection of tissue fibrosis/atrophy and assessment of the extent of fibrosis/atrophy in a tissue (e.g., in a transplanted organ such as a kidney) in a mammal. Early diagnosis of patients with tissue fibrosis/atrophy can help clinicians determine appropriate treatments for those patients. For example, a clinician who diagnoses a patient with progressive fibrosis of transplanted tissue can treat that patient with medication (e.g., anti-fibrotic therapeutic agents or immunosuppressants) that suppresses ongoing tissue injury and fibrosis.

This document is based in part on the identification of molecular features that describe the extent of IFTA in renal allografts, which can be applied to other solid organ transplants and other disease states in which fibrosis is a feature (e.g., cirrhosis of the liver, pulmonary fibrosis, chronic obstructive pulmonary disease (COPD), chronic kidney failure, retroperitoneal fibrosis, cystic fibrosis, mediastinal fibrosis, myelofibrosis, and endomyocardial fibrosis). As described herein, experiments were conducted to determine whether gene expression arrays can distinguish EFTA in renal allograft biopsies performed for clinical indications. A unique set of transcripts was identified that correlated with pathologic features of IFTA and with graft survival. The IFTA pathogenesis based transcript set (IFTA PBT) includes 25 transcripts (referred to herein as IFTAs), any or all of which can be used to distinguish between categories of fibrosis and to detect the severity of fibrosis. This can be applied to renal transplants where IFTA is a presenting feature, and also to other disease states where fibrosis and atrophy are features, including, without limitation, those listed herein. The top five fibrosis transcripts are sufficient to retain the diagnostic power of the gene set.

The identification of an IFTA PBT provides a robust quantitative measure of the degree of interstitial fibrosis and tubular atrophy in allograft biopsies, and adds significant diagnostic value to the limited diagnostic methods and information provided by histopathology. The IFTA PBT represents a new diagnostic classification system and gene expression-based platform to assess tissue fibrosis and allograft deterioration. The IFTA PBT also provides potential therapeutic products, as these transcripts likely identify drug targets. This is of particular interest, as no anti-IFTA therapy is currently available, and interstitial fibrosis/atrophy is the common end stage of numerous diseases. In addition, this technology offers a valuable opportunity to define rejection mechanism(s), revise and develop new end points for clinical trials, and develop new monitoring and diagnostic systems that could be applied to blood, urine and tissue specimens. This gene set information also has applications to other chronic diseases in which fibrosis/atrophy is an element, including cirrhosis of the liver, pulmonary fibrosis, COPD, chronic kidney failure, retroperitoneal fibrosis, cystic fibrosis, mediastinal fibrosis, myelofibrosis, and endomyocardial fibrosis.

The IFTA PBT includes nucleic acids that are differentially expressed in kidney biopsies with IFTA vs. normal kidneys (e.g., those without IFTA). The levels of these nucleic acids and/or polypeptides encoded by these nucleic acids can be used to determine whether tissue transplanted into a mammal has fibrosis, and to determine the extent and type of that fibrosis. For example, transplanted kidney tissue containing cells expressing one or more of the nucleic acids listed in Table 1 at a level that is higher than the average level observed in normal kidney cells (e.g., cells from non-fibrotic areas) can be classified as being tissue with fibrosis/atrophy. In some cases, for example, transplanted tissue containing cells expressing one fifth or more (e.g., one third or more) of the polypeptides encoded by nucleic acids listed in Table 1 at a level that is higher than the average level observed in normal kidney cells can be classified as being fibrotic (e.g., as having fibrosis and atrophy). The levels of multiple nucleic acids or polypeptides can be detected simultaneously using nucleic acid or polypeptide arrays, for example.

In one aspect, this document features a method for detecting tissue fibrosis/atrophy, the method comprising determining whether or not a tissue sample from a human contains cells having a human IFTA profile, wherein the presence of the cells indicates the presence of fibrosis/atrophy in the tissue sample, and wherein the absence of the cells indicates the absence of fibrosis/atrophy. The tissue can be kidney tissue, lung tissue, liver tissue, or heart tissue. The fibrosis can be associated with cirrhosis of the liver, pulmonary fibrosis, COPD, chronic kidney failure, retroperitoneal fibrosis, cystic fibrosis, mediastinal fibrosis, myelofibrosis, endomyocardial fibrosis, or another condition where fibrosis and atrophy are present. The determining step can comprise analyzing nucleic acids or analyzing polypeptides.

In another aspect, this document features a method for assessing tissue fibrosis/atrophy, the method comprising determining the mean expression of IFTAs in cells from tissue in a human, wherein a greater difference between the mean expression of IFTAs and the mean of corresponding reference levels indicates a greater extent of fibrosis/atrophy. The tissue can be kidney tissue, lung tissue, liver tissue, or heart tissue. The fibrosis can be associated with cirrhosis of the liver, pulmonary fibrosis, COPD, chronic kidney failure, retroperitoneal fibrosis, cystic fibrosis, mediastinal fibrosis, myelofibrosis, endomyocardial fibrosis, or another condition where fibrosis and atrophy are present. The determining step can comprise analyzing nucleic acids or analyzing polypeptides.

In another aspect, this document features a method for detecting tissue fibrosis/atrophy and progressive rejection, the method comprising determining whether or not tissue transplanted into a human contains cells having a human IFTA profile, wherein the presence of the cells indicates the presence of fibrosis/atrophy and progressive rejection. The tissue can be kidney tissue, liver tissue, lung tissue, or heart tissue. The tissue can be a kidney, a liver, a lung, or a heart. The method can comprise using kidney cells, liver cells, lung cells, or heart cells obtained from a biopsy to assess the presence or absence of the human IFTA profile. The determining step can comprise analyzing nucleic acids or analyzing polypeptides.

In still another aspect, this document features a method for assessing tissue fibrosis/atrophy, the method comprising determining the mean expression of IFTAs in cells from tissue transplanted into a human, wherein a greater difference between the mean expression of IFTAs and the mean of corresponding reference levels indicates a greater extent of fibrosis/atrophy. The tissue can be kidney tissue, liver tissue, lung tissue, or heart tissue. The tissue can be a kidney, a liver, a lung, or a heart. The method can comprise using kidney cells, liver cells, lung cells, or heart cells obtained from a biopsy to determine the mean expression of IFTAs. The determining can comprise analyzing nucleic acids or analyzing polypeptides.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of different approaches toward histological assessment of inflammation in renal allografts. Relative scoring of inflammation in unscarred areas obeys the current Banff consensus for diagnosing T cell mediated rejection. Absolute scoring of all cortical inflammation independent of type and localization follows the recently introduced and currently evaluated up-dated Banff rules.

FIGS. 2A and 2B are graphs illustrating time dependent changes of infiltrates in biopsies for cause (e.g., for clinical indication). Ordering all 129 biopsies for cause according to time post transplantation (FIG. 2A) illustrates how the early domination of i-Banff (e.g., inflammation in non-fibrotic areas) is giving way to i-IFTA (e.g., inflammation in IFTA areas) as the predominant histological finding with advance time post transplantation. Comparing early (<6 months post TX) versus late (>6 months post TX) allograft biopsies (FIG. 2B) shows a significant increase of fibrosis/atrophy, i-IFTA and nodular infiltrates with time, while the unscarred compartment decreases but with a stable inflammatory burden.

FIGS. 3A and 3B are illustrations of IFTA and inflammation in biopsies for cause and allograft survival. Kaplan-Meier curves show that allografts with fibrosis/atrophy lacking considerable inflammation in this compartment have better outcome than those with extensively inflamed fibrosis/atrophy (FIG. 3A). FIG. 3B shows that inflammation in either cortical compartment (unscarred areas and fibrosis/atrophy) above the current Banff threshold for rejection (i.e., >25%) is associated with an inferior prognosis compared to allografts with infiltrates below this threshold. Events are defined as either allograft loss with return to dialysis or persistent (>3 months) low (<30 ml/min) estimated glomerular filtration rate (eGFR).

FIGS. 4A and 4B are graphs plotting the correlation between individual genes and histological lesions and their overlap. None of the 493 transcripts correlating with tubulitis or i-Banff overlapped the 242 transcripts correlating with fibrosis/atrophy or i-IFTA (FIG. 4A). Considerable overlap was seen between i-Banff and the Banff t-score and between i-IFTA and fibrosis/atrophy. At an arbitrary threshold (r>0.4 and p<0.001), 484 genes were associated with i-Banff, 249 with Banff t-score, 202 with i-IFTA, 172 with fibrosis/atrophy, 34 with nodular infiltrates, and none with perivascular infiltrates (FIG. 4B). i-Banff showed the largest enrichment of cytotoxic T lymphocyte-associated transcripts [CATs (see, U.S. patent application publication nos. 2006/0269948 and 2006/0269949); 35%] and gamma-interferon dependent and rejection-induced transcripts [GRITs (see U.S. patent application publication no. 2006/0269949); 14%), followed by macrophage associated transcripts (19%), and injury-and-repair induced transcripts [IRITs (see U.S. patent application publication no. 2006/0269949); 10%). Only 18% of the correlated transcripts were not annotated as PBTs. For i-IFTA and fibrosis/atrophy, the majority of associated transcripts were not annotated as PBTs. Most of the previously annotated transcripts were B cell associated transcripts (BATs) or immunoglobulin transcripts (IGTs; 23% of probesets for i-IFTA and 16% for fibrosis/atrophy). As a kind of positive control, nodular infiltrates showed the strongest association with BATs/IGTs.

FIG. 5 is a graph plotting the top four mast cell associate transcripts (refer to Table 1) and allograft survival. Kaplan-Meier curves show that within allografts with fibrosis/atrophy (at least Banff grade I) those with high expression of mast cell associated transcripts have a worse prognosis. Events are defined as either allograft loss with return to dialysis or persistent (>3 months) low (<30 ml/min) eGFR.

### DETAILED DESCRIPTION

This document provides methods and materials related to assessing tissue fibrosis and atrophy (e.g., fibrosis and atrophy induced by organ rejection), and develops IFTA as a correlate of progressive functional deterioration. For example, methods and materials are provided herein that can be used to identify a mammal (e.g., a human) as having transplanted tissue that is developing fibrosis and atrophy, which can occur, for instance, with chronic rejection. A human can be identified as having tissue that is undergoing fibrosis/atrophy (e.g., fibrosis/atrophy associated with conditions such as cirrhosis of the liver, pulmonary fibrosis, COPD, chronic kidney failure, retroperitoneal fibrosis, cystic fibrosis, mediastinal fibrosis, myelofibrosis, and endomyocardial fibrosis) if it is determined that the tissue in the human contains cells having a human IFTA profile. In some embodiments, a human can be identified as having tissue undergoing fibrosis/atrophy if it is determined that the tissue in the human contains cells having a mean human IFTA profile. Further, the methods and materials provided herein can be used to identify a mammal (e.g., a human) as having transplanted tissue that is undergoing chronic rejection. A human can be identified as having transplanted tissue that is being chronically rejected if it is determined that the transplanted tissue in the human contains cells having a human IFTA profile. In some cases, a human can be identified as having transplanted tissue that is being chronically rejected if it is determined that the transplanted tissue in the human contains cells having a mean human IFTA profile.

The term “human IFTA profile” as used herein refers to a nucleic acid or polypeptide profile in a sample (e.g., a sample of transplanted tissue) where one or more than one (e.g., 1, 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in Table 1 is present at an elevated level. In some embodiments, a sample identified as having a “human IFTA profile” can refer to a nucleic acid or polypeptide profile in a sample where one fifth or more (e.g., one fourth or more, one third or more, or one half or more) of the nucleic acids or polypeptides encoded by the nucleic acids listed in Table 1 are present at an elevated level. For example, a human IFTA profile can be a nucleic acid or polypeptide profile in a sample where 20%, 24%, 28%, 32%, 36%, 40%, 44%, 48%, 52%, 56%, 60%, 64%, 68%, 72%, 76%, 80%, 84%, 88%, 92%, 96%, or 100% of the nucleic acids or polypeptides encoded by the nucleic acids listed in Table 1 are present at an elevated level.

The term “mean human IFTA profile” as used herein refers to a nucleic acid or polypeptide profile in a sample where the mean expression level of more than one (e.g., 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in Table 1 is elevated. In some embodiments, a sample identified as having a “mean human IFTA profile” can refer to a nucleic acid or polypeptide profile in a sample where the mean expression level of one third or more of the nucleic acids or polypeptides encoded by the nucleic acids listed in Table 1 is elevated. For example, a mean human IFTA profile can be a nucleic acid or polypeptide profile in a sample where the mean expression level of 20%, 24%, 28%, 32%, 36%, 40%, 44%, 48%, 52%, 56%, 60%, 64%, 68%, 72%, 76%, 80%, 84%, 88%, 92%, 96%, or 100% of the nucleic acids or polypeptides encoded by the nucleic acids listed in Table 1 is elevated.

The methods and materials provided herein can be used to predict or detect tissue fibrosis/atrophy in any mammal, such as a human, monkey, horse, dog, cat, cow, pig, mouse, or rat. In addition, the methods and materials provided herein can be used to detect fibrosis/atrophy of any suitable type of transplanted tissue including, without limitation, kidney, heart, liver, pancreas, and lung tissue. For example, the methods and materials provided herein can be used to determine whether or not a human who received a kidney transplant is developing fibrosis/atrophy and chronically rejecting that transplanted kidney, and to what degree that fibrosis/atrophy and chronic rejection is occurring. As another example, the methods and materials provided herein can be used to determine whether or not a human is developing fibrosis/atrophy in an organ due to another disease state (e.g., pulmonary fibrosis, cirrhosis of the liver, COPD, chronic kidney failure, retroperitoneal fibrosis, cystic fibrosis, mediastinal fibrosis, myelofibrosis, or endomyocardial fibrosis), and to what degree the fibrosis/atrophy is occurring.

Any type of sample containing cells can be used to determine whether or not fibrosis/atrophy is present in tissue that has been transplanted into a mammal. For example, biopsy (e.g., punch biopsy, aspiration biopsy, excision biopsy, needle biopsy, or shave biopsy), tissue section, lymph fluid, and blood samples can be used. In some cases, a tissue biopsy sample can be obtained directly from the transplanted tissue or diseased organ. In some cases, a lymph fluid sample can be obtained from one or more lymph vessels that drain from the transplanted tissue or diseased organ.

The term “elevated level” as used herein with respect to the level of a nucleic acid or polypeptide encoded by a nucleic acid listed in Table 1 is any level that is greater than a reference level for that nucleic acid or polypeptide. The term “reference level” as used herein with respect to a nucleic acid or polypeptide encoded by a nucleic acid listed in Table 1 is the level of that nucleic acid or polypeptide typically expressed by cells in tissues that are free of rejection (e.g., chronic rejection) and fibrosis/atrophy. For example, a reference level of a nucleic acid or polypeptide can be the average expression level of that nucleic acid or polypeptide, respectively, in cells isolated from kidney tissue that has not been transplanted into a mammal or that is not undergoing fibrosis/atrophy. Any number of samples can be used to determine a reference level. For example, cells obtained from one or more healthy mammals (e.g., at least 5, 10, 15, 25, 50, 75, 100, or more healthy mammals) can be used to determine a reference level. It will be appreciated that levels from comparable samples are used when determining whether or not a particular level is an elevated level. For example, levels from one type of cells are compared to reference levels from the same type of cells. In addition, levels measured by comparable techniques are used when determining whether or not a particular level is an elevated level.

An elevated level of a nucleic acid or polypeptide encoded by a nucleic acid listed in Table 1 can be any level provided that the level is greater than a corresponding reference level for that nucleic acid or polypeptide. For example, an elevated level of a nucleic acid or polypeptide encoded by a nucleic acid listed in Table 1 can be 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times greater than the reference level for that nucleic acid or polypeptide, respectively. In addition, a reference level can be any amount. For example, a reference level can be zero. In this case, any level greater than zero would be an elevated level.

Any appropriate method can be used to determine the level of a nucleic acid or polypeptide encoded by a nucleic acid listed in Table 1 in a sample. For example, quantitative PCR, in situ hybridization, or microarray technology can be used to measure the level of a nucleic acid listed in Table 1. In some cases, polypeptide detection methods, such as immunochemistry techniques, can be used to measure the level of a polypeptide encoded by a nucleic acid listed in Table 1. For example, antibodies specific for a polypeptide encoded by a nucleic acid listed in Table 1 can be used to determine the level of the polypeptide in a sample.

Once the level of a nucleic acid or polypeptide encoded by a nucleic acid listed in Table 1 is determined in a sample from a mammal, the level can be compared to a reference level for that nucleic acid or polypeptide and used to assess tissue fibrosis in the mammal. For example, a level of one or more than one nucleic acid or polypeptide encoded by a nucleic acid listed in Table 1 that is higher in a sample from a mammal than the corresponding one or more than one reference level can indicate that the mammal comprises transplanted tissue having fibrosis and chronic rejection. In some cases, the presence of one fifth or more of the nucleic acids or polypeptides encoded by the nucleic acids listed in Table 1 at levels higher than the corresponding reference levels in a sample from a mammal can indicate that the mammal comprises transplanted tissue having fibrosis/atrophy associated with, for example, chronic rejection of transplanted tissue, cirrhosis of the liver, pulmonary fibrosis, COPD, chronic kidney failure, retroperitoneal fibrosis, cystic fibrosis, mediastinal fibrosis, myelofibrosis, or endomyocardial fibrosis.

In some cases, the mean (e.g., geometric mean) of the expression levels of more than one nucleic acid or polypeptide encoded by a nucleic acid listed in Table 1 in a sample from a mammal can be used to assess the extent of fibrosis/atrophy (e.g., IFTA and chronic rejection or potential of progressing to IFTA) of a tissue in the mammal. For example, a mean expression level of CPA3 and TPSB2 can be compared to the mean of reference levels of CPA3 and TPSB2 to assess the extent of fibrosis of a tissue in the mammal. In some embodiments, the mean of the expression levels of one fifth or more (e.g., 20%, 24%, 28%, 32%, 36%, 40%, 44%, 48%, 52%, 56%, 60%, 64%, 68%, 72%, 76%, 80%, 84%, 88%, 92%, 96%, or 100%) of the nucleic acids or polypeptides encoded by the nucleic acids listed in Table 1 in a sample from a mammal can be used to assess the extent of fibrosis/atrophy of a tissue in the mammal. Such a mean expression level in a sample from a mammal (e.g., a mammal having transplanted tissue) can be compared to the mean of corresponding reference levels. The greater the difference between the mean of the expression levels of more than one nucleic acid or polypeptide encoded by a nucleic acid listed in Table 1 and the mean of corresponding reference levels, the greater the extent of IFTA and potential for progression to chronic rejection.

In some cases, the value of the mean of the expression levels of more than one nucleic acid listed in Table 1 (e.g., at least one third of the nucleic acids listed in Table 1, or all of the nucleic acids listed in Table 1) can be inserted into an equation defining a standard curve to estimate the IFTA burden in a sample from a mammal. A standard curve can be generated by analyzing a series of dilutions of an RNA sample obtained from renal cells from one or more healthy donors. The RNA sample can be diluted into increasing amounts of RNA isolated from a nephrectomy sample from a mammal free of tissue fibrosis/atrophy. Each sample in the dilution series can be analyzed to determine the expression levels of more than one nucleic acid listed in Table 1 (e.g., at least one third of the nucleic acids listed in Table 1, or all of the nucleic acids listed in Table 1), and the mean expression level can be plotted against the dilution factor of the RNA sample. The mean expression level of the same nucleic acids used to generate a standard curve in a sample from a mammal can then be inserted into the equation defining the standard curve, and the equation can be solved for the dilution of the IFTA RNA sample to estimate the IFTA burden in the sample from the mammal. An estimated IFTA burden in a sample from a mammal that is higher than a corresponding reference value can indicate that transplanted tissue in the mammal is being rejected, or is susceptible to being rejected and progressing with IFTA. A reference value can be, for example, an average of estimated IFTA burden values in more than one corresponding control sample obtained from more than one mammal that does not have transplanted tissue.

In some cases, the expression level of one nucleic acid or polypeptide encoded by a nucleic acid listed in Table 1 in a sample from a mammal can be used to assess the extent of fibrosis/atrophy of a tissue in the mammal. The expression level of the nucleic acid or polypeptide encoded by a nucleic acid listed in Table 1 can be compared to the corresponding reference level. The greater the difference between the expression level of the nucleic acid or polypeptide encoded by a nucleic acid listed in Table 1 and the corresponding reference level, the greater the extent of fibrosis/atrophy and, in the case of transplantation, the greater the state of or potential for chronic rejection.

In the case of tissue transplantation, the methods and materials provided herein can be used at any time following transplant to determine whether or not the transplanted tissue will develop fibrosis/atrophy (e.g., IFTA). For example, a sample obtained from transplanted tissue at any time following the tissue transplantation can be assessed for the presence of cells expressing an elevated level of one or more nucleic acids or polypeptides encoded by nucleic acids provided herein. In some cases, a sample can be obtained from transplanted tissue 1, 2, 3, 4, 5, 6, 7, 8, 9, 10, 11, 12, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 35, 40, 45, 50, 55, 60, or more days) after the transplanted tissue was transplanted. For example, a sample can be obtained from transplanted tissue 2 to 7 days (e.g., 4 to 6 days) after transplantation and assessed for the presence of cells expressing an elevated level of a nucleic acid or polypeptide encoded by a nucleic acid provided herein. Typically, a biopsy can be obtained any time after transplantation if a patient experiences reduced graft function.

This document also provides methods and materials to assist medical or research professionals in determining whether or not a mammal has fibrosis/atrophy (e.g., IFTA associated with chronic tissue rejection). Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more nucleic acids or polypeptides encoded by nucleic acids listed in Table 1 in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides nucleic acid arrays. The arrays provided herein can be two-dimensional arrays, and can contain at least two different nucleic acid molecules (e.g., at least three, at least five, at least ten, at least 20, at least 30, at least 40, at least 50, or at least 60 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In some cases, an array can contain one or more cDNA molecules encoding, for example, partial or entire polypeptides. In addition, each nucleic acid molecule can have any sequence. For example, the nucleic acid molecules of the arrays provided herein can contain sequences that are present within nucleic acids listed in Table 1.

In some cases, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid listed in Table 1. For example, an array can contain 60 nucleic acid molecules located in known positions, where each of the 60 nucleic acid molecules is 100 nucleotides in length while containing a sequence that is (1) 90 nucleotides is length, and (2) 100 percent identical, over that 90 nucleotide length, to a sequence of a nucleic acid listed in Table 1. A nucleic acid molecule of an array provided herein can contain a sequence present within a nucleic acid listed in Table 1 where that sequence contains one or more (e.g., one, two, three, four, or more) mismatches.

The nucleic acid arrays provided herein can contain nucleic acid molecules attached to any suitable surface (e.g., plastic, nylon, or glass). In addition, any appropriate method can be used to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

This document also provides arrays for detecting polypeptides. The arrays provided herein can be two-dimensional arrays, and can contain at least two different polypeptides capable of detecting polypeptides, such as antibodies (e.g., at least three, at least five, at least ten, at least 20, at least 30, at least 40, at least 50, or at least 60 different polypeptides capable of detecting polypeptides). The arrays provided herein also can contain multiple copies of each of many different polypeptides. In addition, the arrays for detecting polypeptides provided herein can contain polypeptides attached to any suitable surface (e.g., plastic, nylon, or glass).

A polypeptide capable of detecting a polypeptide can be naturally occurring, recombinant, or synthetic. The polypeptides immobilized on an array also can be antibodies. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a mouse, chicken, human, rabbit, sheep, or goat antibody. Such an antibody can be capable of binding specifically to a polypeptide encoded by a nucleic acid listed in Table 1. The polypeptides immobilized on the array can be members of a family such as a receptor family.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a nucleic acid encoding the partial antibody sequence. In some cases, an antibody fragment can be enzymatically or chemically produced by fragmentation of an intact antibody. In addition, numerous antibodies are available commercially. An antibody directed against a polypeptide encoded by a nucleic acid listed in Table 1 can bind the polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

### EXAMPLES

### Example 1

### Materials and Methods

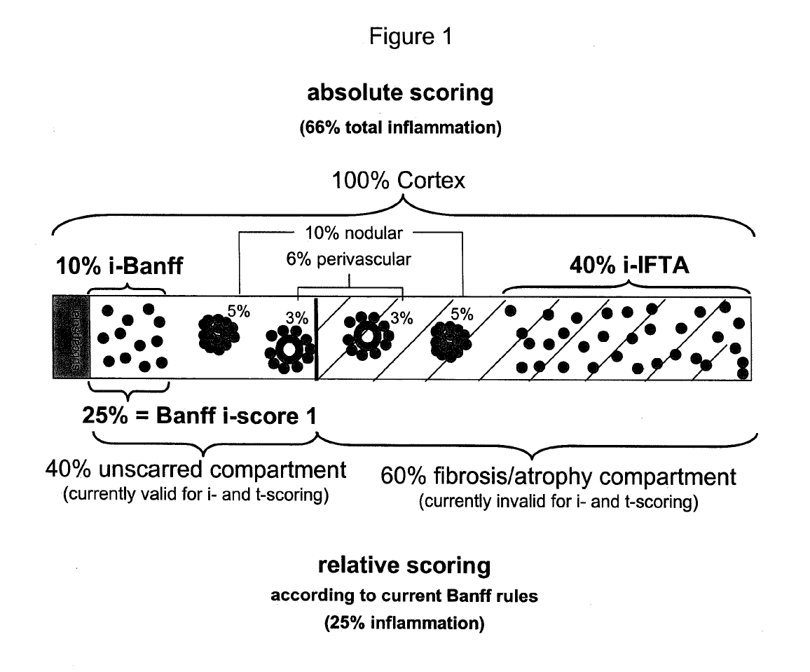

Biopsies and histopathological scoring: A “training set” of 129 clinically indicated renal allograft biopsies was obtained from 104 consenting patients. Biopsies were taken between 1 week and 20 years post transplant, with a median of 19 months. For the present studies, histopathological re-evaluation of the 129 biopsies was done by one observer. All samples fulfilled the minimal criteria for adequacy and were stained (including C4d on frozen sections) and scored according to the current Banff classification (Racusen et al. (1999) Kidney Int 55:713-723; Solez et al. (2008) Am J Transplant 8:753-760; and Racusen et al. (2003) Am J Transplant 3:708-714). In addition, the extent of four different types of interstitial infiltrates was semi-quantitatively assessed in the renal cortex: 1) infiltrates in unscarred areas (i-Banff); 2) infiltrates in areas of fibrosis/atrophy (i-IFTA); 3) nodular infiltrates; and 4) perivascular infiltrates (Mengel et al. (2007) Am J Transplant 7:356-365). Analogously, the extent of fibrosis/atrophy was assessed independent of whether or not it was inflamed. In contrast to the current Banff rules, these histological features were assessed as absolute percentages related to the whole cortex as 100% (FIG. 1), to make the numbers for their extent comparable. For validation purposes, another 50 biopsies for cause obtained from 50 patients (after the above-described 129 biopsies) were analyzed. This “validation set” included 17 biopsies from a different center (University of Illinois at Chicago).

Microarray experiments: An additional 18-gauge biopsy core was collected for gene expression analysis. The tissue was placed immediately in RNALater (Qiagen, Valencia, Calif.) and stored at −20° C. RNA extraction, labeling, and hybridization to the HG_U133_Plus_{—}2.0 GeneChip (Affymetrix, Santa Clara, Calif.) were carried out according to the manufacturer's protocols (available on the World Wide Web at affymetrix.com). Microarrays were scanned using a GeneArray Scanner and processed with GeneChip Operating Software Version 1.4.0 (both from Affymetrix). Microarray data were pre-processed by robust multi-array analysis (RMA) and implemented in Bioconductor version 2.2., and fold changes were calculated relative to native kidney samples taken from unaffected areas of the cortex of eight tumor nephrectomies.

Pathogenesis Based Transcript sets: A system was developed for collapsing large scale genome wide expression data into pathogenesis based transcript sets (PBTs). This system was used to show the utility of these gene sets for diagnosing rejection in renal transplant biopsies (Mueller (2007) Am J Transplant 7:2712-2722).

Thus biopsies included in the present study were part of a larger data set, where PBT results were analyzed in relationship to Banff scores and clinical diagnosis of rejection. PBTs reflect the major biological processes in allografts: cytotoxic T cell associated transcripts (CATs; Einecke et al. (2005) Am J Transplant 5:1827-1836; and Einecke et al. (2006) Am J Transplant 6Suppl 2:401-402), interferon-K dependent rejection induced transcripts (GRITs; Einecke et al. (2006) Am J Transplant 6Suppl 2:401-402; and Famulski et al. (2006) Am J Transplant 6:1342-1354), kidney transcripts with decreased expression during rejection (KTs; Einecke et al. (2007) Am J Transplant 7:1121-1130), injury and repair induced transcripts (IRITs; Famuiski et al. (2007) Am J Transplant 7:2483-2495), immunoglobulin transcripts (IGTs; Einecke et al. (2008) Am J Transplant, in press), and B cell associated transcripts (BATs; Einecke et al. (2008) Am J Transplant, in press). Microarray gene expression results were collapsed into PBT scores as the geometric mean of fold changes across all probe sets in that PBT. Fold changes were defined as the ratio of expression values in a biopsy to the average value from eight native kidney control samples. Probe sets in each previously published PBT are available online at transplants.med.ualberta.ca. The PBT annotation of a probe set thus provided a rapid way of understanding the biological processes represented by changes in that probe set.

Immunohistochemistry: The following anti-human antibodies were obtained from DAKO (Carpinteria, Calif.) and applied to paraffin sections: anti CD3 (polyclonal), anti CD68 (clone PGMI), anti CD20 (clone L26), anti CD138 (clone MI15), and anti mast cell tryptase (clone AA1). Stains were done on a BENCHMARK® automated stainer (Ventana Medical Systems, Inc.). Sections were pre-treated for epitope retrieval and incubated with primary antibodies, followed by respective biotinylated secondary antibody incubation. Staining was developed using an avidin-biotin-based detection system with peroxidase and DAB visualization (Ventana I-VIEW™ DAB). For each marker, the percentage of stained cells relative to all inflammatory cells was semi-quantitatively assessed for the i-Banff and the i-IFTA compartment.

Statistical analysis: Spearman correlations were used to assess the relationship between the different histological features and gene expression values. Means were compared using a 2-sample t-test. For allograft outcome analysis, patients were followed after biopsy for a mean of 24.6±8.7 months (range=3 to 41 months) and either death-censored or censored for end of follow-up. Patients in the validation set were followed for 11.7±3.2 months (range=1.3 to 16.3 months) after biopsy. An event was defined as either graft loss or persistent low eGFR (estimated by the Cockcroft-Gault formula; Cockcroft and Gault (1976) Nephron 16:31-41) defined as at least three months of eGFR<30 ml/min (time to event=time to end of three months low eGFR). Survival analysis was performed on the last biopsy from each patient using Kaplan Meier analysis with log-rank testing (SPSS 15.0 software; SPSS Inc., Chicago, Ill.).

### Example 2

### Histological Features and Time After Transplantation

All consecutive biopsies for cause were included if adequate for assessment, with the exception of cases with BK virus-associated interstitial nephritis (BK nephropathy). Demographics of the 104 patients providing the 129 biopsies were previously published (Mueller et al. (2007) Am J Transplant 7:2712-2722). By Banff criteria (Solez et al. (2008), supra), 20 biopsies had changes suspicious for rejection (i.e., borderline), 19 had T-cell mediated rejection (TCMR), 14 were C4d positive, had circulating anti-HLA antibodies, and met histological criteria of antibody mediated rejection (2 acute and 12 chronic-active ABMR), and 76 did not have histologic criteria for rejection. Within the validation set, histopathological diagnoses according to Banff were: three ABMR (1 acute and 2 chronic-active), 10 borderline, 8 TCMR, and 29 cases without histologic criteria for rejection.

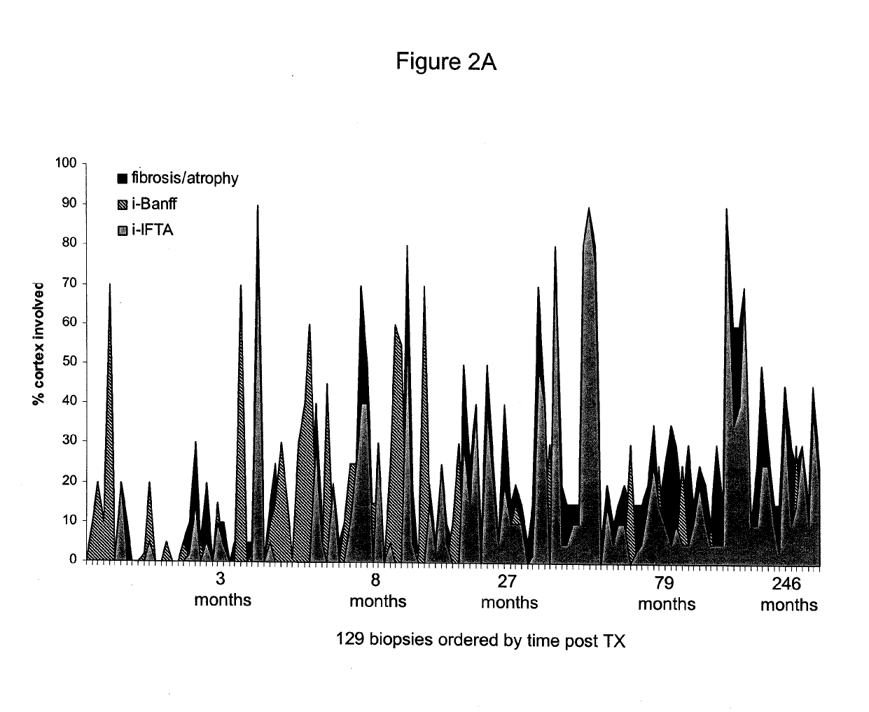

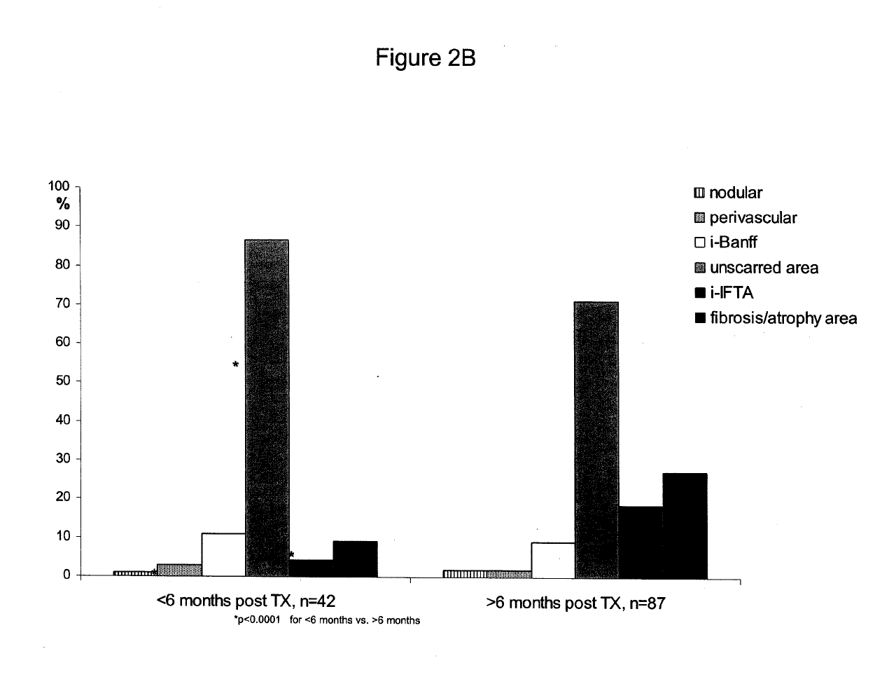

In the training set (n=129), i-IFTA correlated with the extent of fibrosis/atrophy (r=0.911, p<0.0001). In biopsies with fibrosis/atrophy, 0-100% of the fibrosis/atrophy area was inflamed (mean 49.5%; (FIG. 2A). The extent of fibrosis/atrophy as well as the extent of i-IFTA correlated with time after transplantation (r=0.582, p<0.0001; and r=0.554, p<0.0001, respectively), and were greater in biopsies taken more than 6 months after transplantation (FIG. 2B). The percentage of the biopsy showing i-IFTA was greater in biopsies take >6 months post transplant (early vs. late: 26.2% vs. 81.6%, p<0.0001), whereas the extent of i-IFTA in late biopsies still correlated with time post transplant (r=0.25, p=0.021).

The i-Banff did not correlate with the extent of i-IFTA, but correlated strongly with the degree of tubulitis (r=0.85, p<0.0001). The extent of i-Banff negatively correlated with the time elapsed after transplantation (r=−0.27, p=0.014); although the extent of inflammation in the unscarred compartment did not significantly change over time, the unscarred area was less in later biopsies as fibrosis/atrophy increased.

Nodular infiltrates and perivascular infiltrates were present in both compartments, but were quantitatively minor contributors to inflammation in both. Nodular infiltrates increased with time, while perivascular infiltrates did not.

### Example 3

### Inflammation and Fibrosis/Atrophy in Biopsies for Cause and Allograft Survival

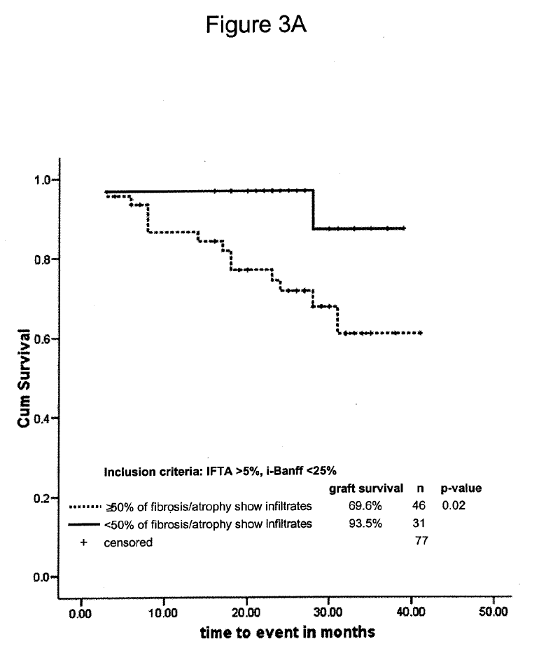

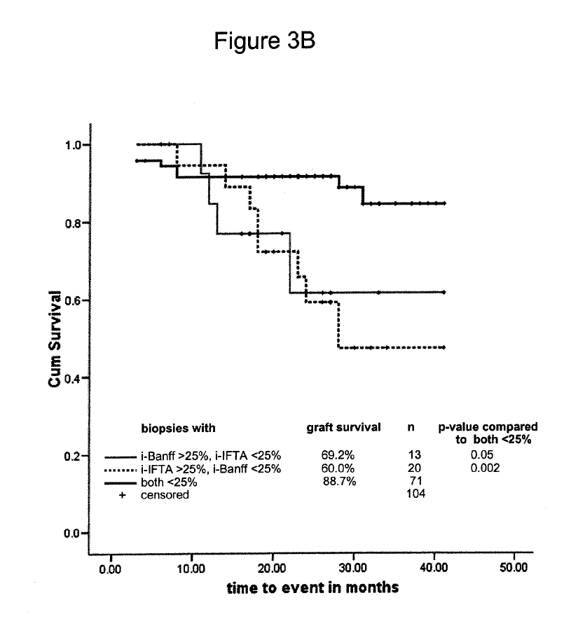

Since fibrosis/atrophy and inflammation in this compartment (i.e., i-IFTA) are highly correlated with each other, studies were conducted to address the question of whether the inflammation in fibrosis/atrophy provides additional information compared to fibrosis/atrophy alone (i.e., whether i-IFTA is relevant to prognosis). The subset of allograft biopsies showing at least grade I IFTA according to Banff (i.e., ≧ci1/ct1) but i-Banff<25% (n=77) was selected (Racusen et al. (1999), supra). These were split into two groups: biopsies where ≧50% of the fibrosis/atrophy compartment was inflamed (n=46), and biopsies where <50% of the fibrosis/atrophy compartment was inflamed (n=31). The high i-IFTA group had a worse prognosis than the low i-IFTA group (FIG. 3A: 93.5% vs. 69.6% survival, p=0.02) indicating that the inflammation in fibrosis/atrophy is relevant.

Outcomes for allografts with predominantly i-Banff (i.e., above the current Banff threshold for rejection; =25% of cortex involved) were compared to those showing predominantly i-IFTA (FIG. 3B). Allografts with >25% of the cortex inflamed either in the unscarred areas (69%, p=0.05) or in the fibrosis/atrophy compartment (60%, p=0.002) had worse survival than those with less than 25% of the two cortical compartments inflamed (89%), indicating that both patterns of inflammation have prognostic relevance.

### Example 4

### Correlations with Individual Genes Confirm Mutually Exclusive Associations of Transcripts in i-Banff/Tubulitis Versus IFTA/i-IFTA

The correlation between gene expression and histological features was examined using 54676 probe sets on the HG_U133_Plus_{—}2.0 GeneChip. A threshold correlation coefficient of r>0.4 and a p value of <0.001 for a probe set were considered to be strongly correlated with a histological feature.

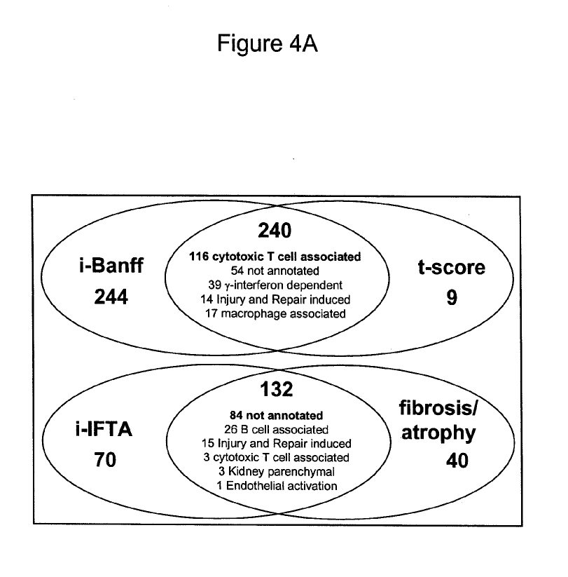

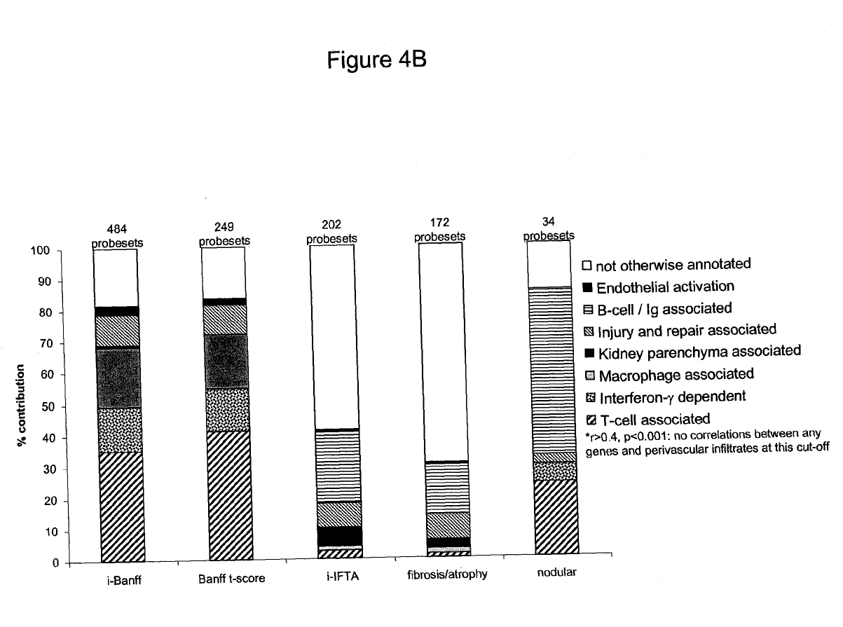

This approach identified 484 probe sets associated with i-Banff, 249 with Banff t-score, 202 with i-IFTA, 172 with fibrosis/atrophy, 34 with nodular infiltrates, and none with perivascular infiltrates. Remarkably, none of the 493 transcripts that were correlated with i-Banff and/or tubulitis overlapped the 242 transcripts that were correlated with fibrosis/atrophy and/or i-IFTA (FIG. 4A).

The PBT annotation of the probe sets correlating with the extent of each histological feature is indicated in FIG. 4B. The extent of i-Banff correlated mostly with CATs (35%) and GRITs (14%), followed by macrophage associated transcripts (19%), and IRITs (10%). Only 18% of the correlated transcripts were not annotated as PBTs. There were no Bcell/plasma cell transcripts associated with i-Banff and/or tubulitis. Considerable overlap (FIG. 4A) of probe sets was observed between i-Banff and tubulitis. Two hundred and forty (240) probe sets were simultaneously correlated with the extent of both lesions.

For fibrosis/atrophy and i-IFTA, most of the annotated transcripts were BATs or IGTs (16% of probe sets for fibrosis/atrophy and 23% for i-IFTA were annotated as BATs or IGTs). More than 50% of the fibrosis/atrophy and/or i-IFTA-associated probe sets were not annotated as PBTs (70% for fibrosis/atrophy and 59% for i-IFTA). Considerable overlap also was present between the degree of fibrosis/atrophy and i-IFTA, with 132 probe sets being shared.

Nodular infiltrates showed the strongest association with BATs/IGTs. Fifty-three percent (53%) of the correlated probe sets were annotated with these PBTs, whereas the most highly correlated probe set was that coding for CD20 (r=0.53). The second largest group of correlated probe sets were annotated as CATs (24%), while 15% had no PBT annotation.

### Example 5

### Fibrosis/Atrophy and i-IFTA Associated Transcripts

Transcripts that were not previously annotated by PBTs but were correlated with the extent of fibrosis/atrophy and/or IFTA were examined. Probe sets not identified by Affymetrix, annotated as PBTs, or coding for hypothetical proteins were eliminated. In cases with multiple probe sets representing the same gene, only the most highly correlated probe set from both overlapping lists was retained. Table 1 shows the 25 genes most strongly correlated with the extent of these two features, i.e., fibrosis/atrophy and/or i-IFTA. Four of the top six genes code for transcripts associated with mast cells: carboxypeptidase A3, mast cell tryptase beta 2, tryptase alpha/beta1, and Fc IgE receptor alpha. With the exception of the probe set for TGFB2 (r=0.404) none of the typical fibrogenesis-associated transcripts (e.g., TGFβ- or collagen-related) correlated with the extent of fibrosis/atrophy or i-IFTA above the arbitrary threshold.

### Example 6

### Confirmation by Immunohistochemistry

A subset of 33 biopsies representing the spectrum of histological features and with paraffin embedded material available were stained. T cells (CD3), histiocytes (CD68), B cells (CD20), plasma cells (CD138), and mast cells (mast cell tryptase) in both inflammatory compartments were studied. The percentage of CD20+ B cells was greater in i-IFTA (8.1±7.4% vs. 3.2±3.8%, p=0.006). The percentage of CD138+ positive interstitial cells (plasma cells) was greater in the i-IFTA compartment, but this difference did not reach statistical significance (8.2±14.1% vs. 4.2±7.1%, p>0.05). The % mast cells was higher in biopsies with fibrosis/atrophy, and was greater in the i-IFTA compartment than in the i-Banff compartment (6.5±4.8% vs. 1.9±1.5%, p=0.0004).

T cells and macrophages were the dominant cell types in both compartments, but the relative frequency of other cell types (B cells, plasma cells, mast cells) was higher in i-IFTA, making the T cell and macrophage percentage relatively lower (i-IFTA vs. i-Banff: T cells 40.8±13.1% vs. 53.2±12.4%, p=0.006; macrophages 17.2±10.1% vs. 27.1±13.3%, p=0.02).

### Example 7

### Mast Cell Associated Transcript Set

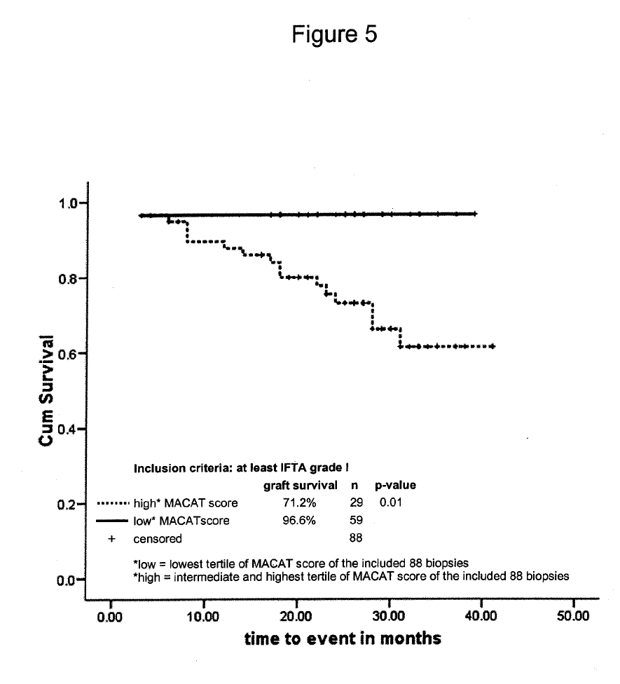

To assess expression of mast cell associated transcripts across all biopsies, the expression values of the four mast cell associated transcripts were collapsed into a “Mast cell associated transcript” (MACAT) score for each biopsy. MACAT scores were higher in biopsies with more fibrosis/atrophy and i-IFTA (<25% vs. ≧25%, p<0.0001), but not in biopsies with more i-Banff. The MACAT score correlated with time after transplantation (r=0.55, p<0.01), extent of i-IFTA (r=0.63, p<0.01) and fibrosis/atrophy (r=0.61, p<0.01), but not i-Banff (r=−0.03, p>0.05). Even in biopsies with at least Banff grade I fibrosis/atrophy (i.e., >5% of cortex involved), MACAT scores still correlated with the extent of i-IFTA (r=0.55, p<0.0001) and fibrosis/atrophy (r=0.51, p<0.0001). Low MACAT scores (i.e., lowest tertile of MACAT scores in all 104 patients) were associated with better allograft survival (94.3%) compared to high (i.e., the intermediate and highest tertile) MACAT scores (73.9%, p=0.01). Restricting this analysis to biopsies with fibrosis/atrophy (at least Banff grade I, n=88), high MACAT scores were still associated with a worse allograft survival (71.2%), compared to those biopsies with fibrosis/atrophy and low MACAT scores (96.6%, p=0.01; FIG. 5).

### Example 8

### Analysis of the Validation Set

In the validation set, three mast cell associated transcripts (carboxypeptidase A3, mast cell tryptase beta 2, tryptase alpha/beta1) were within the top ten probe sets when microarray expression values were correlated with the extent of i-IFTA and fibrosis/atrophy. MACAT scores were higher in biopsies with more fibrosis/atrophy and i-IFTA (<25% vs. ≧25%, p=0.004), and correlated with the extent of fibrosis/atrophy (r=0.72, p<0.0001) and i-IFTA (r=0.65, p<0.0001). In terms of allograft outcome, low MACAT scores (i.e., lowest tertile of MACAT scores in the 50 patients of the validation set) were associated with a better allograft survival (100%) as compared to high MACAT scores (76.5%, p<0.02). Restricting this analysis to biopsies with fibrosis/atrophy (at least Banff grade I, n=40) still showed lower allograft survival for high MACAT scores (70.4%), compared to those biopsies with fibrosis/atrophy and low MACAT scores (100%, p=0.02).

A simple threshold classifier was built from the original set of 129 biopsies, based on MACAT scores. The classifier was designed to predict recovery of allograft function after biopsy. For this purpose, the change in eGFR between biopsy and 6-months post-biopsy was used, and two classes were defined: patients with unchanged or decreasing eGFR (i.e., no recovery of allograft function after biopsy), and patients with increasing eGFR (recovery of function of at least 10% from the value at biopsy). The training set threshold predicted eGFR status in the test set (n=50) with an accuracy of 60%, sensitivity of 82%, specificity of 32%, positive predictive value of 61%, and negative predictive value of 58%. The accuracy was significantly higher than that obtained using randomly shuffled data (56%) in a permutation test (p=0.001). Classifiers based on single training set:test set splits make very inefficient use of the available data (Simon (2006) J Natl Cancer Inst 98:1169-1171). Therefore, a classifier also was built using the full dataset (n=179), and error rates were estimated using leave-one-out cross-validation (LOOCV). This resulted in the following estimates: an accuracy of 71%, sensitivity of 84%, specificity of 47%, positive predictive value of 74%, and negative predictive value of 63%. The accuracy was significantly higher than that obtained using randomly shuffled data (64%) in a permutation test (p=0.001).

TABLE 1

| | | | Corre- |
|---|---|---|---|
| Affymetrix | Gene | | lation* |
| ID | symbol | Gene name | r-value |
| 205624_at | CPA3 | mast cell carboxypeptidase A3 | 0.619 |
| 207134_x_at | TPSB2 | mast cell tryptase beta 2 | 0.566 |
| 204719_at | ABCA8 | ABC transporters-ATP-binding | 0.564 |
| | | cassette, sub-family A (ABC1), | |
| | | member 8 | |
| 205683_x_at | TPSAB1 | mast cell tryptase alpha/beta 1 | 0.555 |
| 205044_at | GABRP | gamma-aminobutyric acid | 0.554 |
| | | (GABA) A receptor | |
| 211734_s_at | FCER1A | receptor for Fc fragment of IgE, | 0.547 |
| | | high affinity I, expressed | |
| | | predominantly on mast cells | |
| 209173_at | AGR2 | anterior gradient homolog 2 | 0.523 |
| 229461_x_at | NEGR1 | neuronal growth regulator 1 | 0.509 |
| 213974_at | ADAMTSL3 | ADAMTS-like 3 | 0.503 |
| 221933_at | NLGN4X | neuroligin 4 | 0.499 |
| 228310_at | ENAH | cytoskeleton regulatory protein | 0.497 |
| | | hMena | |
| 202508_s_at | SNAP25 | synaptosomal-associated protein | 0.497 |
| 226435_at | PAPLN | papilin | 0.486 |
| 228241_at | BCMP11 | AGR3 = anterior gradient | 0.485 |
| | | homolog | |
| 219552_at | SVEP1 | sushi, von Willebrand factor | 0.484 |
| | | type 1, EGF and pentraxin | |
| | | domain containing 1 | |
| 227088_at | PDE5A | phosphodiesterase 5A | 0.479 |
| 219778_at | ZFPM2 | zinc finger protein, multitype 2 | 0.474 |
| 207496_at | MS4A2 | Fc fragment of IGE, high affinity | 0.47 |
| | | I, receptor for; beta polypeptide | |
| 202992_at | C7 | complement component 7 | 0.468 |
| 1558714_at | ROBO1 | roundabout, axon guidance | 0.467 |
| | | receptor, homolog 1 | |
| 219867_at | CHODL | chondrolectin | 0.466 |
| 206336_at | CXCL6 | chemokine (C-X-C motif) ligand | 0.466 |
| | | 6 (granulocyte chemotactic | |
| | | protein 2) | |
| 210258_at | RGS13 | regulator of G-protein signaling | 0.462 |
| | | 13 | |
| 201489_at | PPIF | peptidylprolyl isomerase F | −0.47 |
| | | (cyclophilin F, CYP3; Cyp-D | |
| 2083321_s_at | CABP1 | calcium binding protein 1 | −0.49 |

*Spearman correlation, p < 0.001;
NA = not available;
EMC = extracellular matrix components

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying the presence of tissue fibrosis/atrophy in a human, said method comprising determining whether or not a sample of tissue from said human contains cells having a human interstitial fibrosis and tubular atrophy (IFTA) profile, classifying said tissue as undergoing fibrosis/atrophy if said cells are present, or classifying said tissue as not undergoing fibrosis/atrophy if said cells are absent.

2. The method of claim 1, wherein said tissue is kidney tissue, lung tissue, liver tissue, or heart tissue.

3-5. (canceled)

6. The method of claim 1, wherein said fibrosis is associated with cirrhosis of the liver, pulmonary fibrosis, chronic obstructive pulmonary disease (COPD), chronic kidney failure, retroperitoneal fibrosis, cystic fibrosis, mediastinal fibrosis, myelofibrosis, endomyocardial fibrosis, or another condition where fibrosis and atrophy are present.

7. The method of claim 1, wherein said determining step comprises analyzing nucleic acids.

8. The method of claim 1, wherein said determining step comprises analyzing polypeptides.

9. A method for assessing tissue fibrosis and atrophy, said method comprising measuring the mean expression of IFTAs in cells from human tissue, and determining the difference between said mean expression of IFTAs and the mean of corresponding reference levels, wherein a greater difference indicates a greater extent of fibrosis/atrophy.

10. The method of claim 9, wherein said tissue is kidney tissue, lung tissue, liver tissue, or heart tissue.

11-13. (canceled)

14. The method of claim 9, wherein said fibrosis is associated with cirrhosis of the liver, pulmonary fibrosis, COPD, chronic kidney failure, retroperitoneal fibrosis, cystic fibrosis, mediastinal fibrosis, myelofibrosis, endomyocardial fibrosis, or another condition where fibrosis and atrophy are present.

15. The method of claim 9, wherein said determining step comprises analyzing nucleic acids.

16. The method of claim 9, wherein said determining step comprises analyzing polypeptides.

17. A method for identifying the presence of tissue fibrosis/atrophy and progressive rejection in a human, said method comprising determining whether or not tissue transplanted into a human contains cells having a human IFTA profile, and classifying said tissue as undergoing fibrosis/atrophy and progressive rejection if said cells are present, or classifying said tissue as not undergoing fibrosis/atrophy and progressive rejection if said cells are absent.

18. The method of claim 17, wherein said tissue is kidney tissue, liver tissue, lung tissue, or heart tissue.

19. The method of claim 17, wherein said tissue is a kidney, a liver, a lung, or a heart.

20. The method of claim 17, wherein said method comprises using kidney cells, liver cells, lung cells, or heart cells obtained from a biopsy to assess the presence or absence of said human IFTA profile.

21. The method of claim 17, wherein said determining step comprises analyzing nucleic acids.

22. The method of claim 17, wherein said determining step comprises analyzing polypeptides.

23. A method for assessing tissue fibrosis/atrophy, said method comprising measuring the mean expression of IFTAs in cells from tissue transplanted into a human, and determining the difference between said mean expression of IFTAs and the mean of corresponding reference levels, wherein a greater difference indicates a greater extent of fibrosis/atrophy.

24. The method of claim 23, wherein said tissue is kidney tissue, liver tissue, lung tissue, or heart tissue.

25. The method of claim 23, wherein said tissue is a kidney, a liver, a lung, or a heart.

26. The method of claim 23, wherein said method comprises using kidney cells, liver cells, lung cells, or heart cells obtained from a biopsy to determine said mean expression of IFTAs.

27. The method of claim 23, wherein said determining comprises analyzing nucleic acids.

28. The method of claim 23, wherein said determining comprises analyzing polypeptides.
